(19) United States
(12) Patent Application Publication
(10) Pub. No.: US 2006/0288906 A1
(43) Pub. Date: Dec. 28, 2006

(54) Process of preparation of specific color effect pigments

(76) Inventors: Martin Wulf, Langenfeld (DE); Klaus Taennert, Wuppertal (DE); Dirk Allard, Radevormwald (DE); Rudolf Zentel, Nierstein (DE)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/410,399

(22) Filed: Apr. 25, 2006

Related U.S. Application Data
(60) Provisional application No. 60/675,220, filed on Apr. 27, 2005.

(51) Int. Cl. C09D 11/00; A61K 8/02

(52) U.S. Cl. 106/31.9; 424/401

(57) ABSTRACT

The invention provides a process of preparation photonic crystals which is distinguished by high efficiency and which provide photonic crystals having an opalescent effect of high quality; the process comprises (a) providing a dispersion of mono-disperse spheres in a liquid, (b) applying the dispersion onto a plain surface of an absorbent substrate selected from the group consisting of paper, textile and wood, and (c) removing the liquid from the dispersion on the surface to produce the photonic crystals in the form of a tightly packed and regularly arranged structure of mono-disperse spheres in the resulted particles; using the process according to the invention, it is possible to produce photonic crystals usable as pigment particles with a high optical brilliance in the color and with a color stability for the use in, e.g., coating compositions and inks.

### CROSS-REFERENCE TO RELATED APPLICATION

This application claims the benefit of U. S. Provisional Application Ser. No. 60/675,220 filed on Apr. 27, 2005 which is hereby incorporated by reference in its entirety.

### FIELD OF THE INVENTION

The invention relates to a process of preparation of color effect pigments, which are photonic crystals usable as interference pigments to provide a more efficient preparation procedure.

### BACKGROUND OF THE INVENTION

Various types of pigments may be usable such as simple color pigments, mica pigments, special effect pigments for decorative purposes, e.g., for coating substrate surfaces and pigmentation of printing paints.

Novel pigments have been found which resemble naturally occurring opals and wherein the color activity is produced by Bragg diffraction of the incident light on the grid planes of spheres arranged in a crystal-like manner on the substrate surface (the spheres diffract the light according to Bragg's law). Artificial opals may be synthesized by self-assembling of mono-disperse mano-scaled particles which are mono-disperse spheres.

WO 01/88044 describes pigments with an opalescent effect. The pigment particles consist of mono-disperse spheres in a three-dimensional, highly packed structure, also called three-dimensional photonic crystals, with a diameter of 50 nm to 2 μm. The pigments may be prepared by dispersing the mono-disperse spheres in a liquid medium, applying the dispersion on a smooth surface, removing the liquid medium and separating the particles from the surface. The smooth surface may be a surface of metal, semiconductor, glass or plastic substrates.

According to WO 03/058299 flexible substrates, such as, polyester films and metal stripes as well as inflexible substrates, such as, glass or metal plates may be used for the preparation of the specific colorant particles.

In EP-A 1184195, an ink jet method is described wherein the ink jet recording material, such as, paper or plastic comprises a top layer containing core-shell particles of refractive nature.

The preparation of the above described pigment particles in form of an ordered array of the particles on the substrate surface needs a time period of typically at least one hour to several hours, and this process affects the whole preparation procedure.

### SUMMARY OF THE INVENTION

The invention provides a process of preparation photonic crystals which is distinguished by high efficiency and which provides photonic crystals having an opalescent effect of high quality.

The process according the invention provides a preparation process of photonic crystals comprising:
(a) providing a dispersion of mono-disperse spheres in a liquid,
(b) applying the dispersion onto a plain surface of an absorbent substrate selected from the group consisting of paper, textile and wood, and
(c) removing the liquid from the dispersion on the surface to produce the photonic crystals in the form of a tightly packed and regularly arranged structure of mono-disperse spheres in the resulted particles.

Using the process according to the invention, it is possible to produce photonic crystals usable as pigment particles with a high optical color brilliance and with a color stability for the use in, e.g., coating compositions and inks. The process according to the invention provides an efficient procedure of preparation of the photonic crystals by reducing the preparation time enormously in a range of 10 seconds to 5 minutes at room temperature, preferably less than 1 minute.

### BRIEF DESCRIPTION OF DRAWINGS

FIG. 1 shows a side view of a photonic crystal prepared according to Example 2.

FIG. 2 shows a top view of a photonic crystal after drying.

### DETAILED DESCRIPTION

These and other features and advantages of the present invention will be more readily understood by those of ordinary skill in the art from a reading of the following detailed description. It is to be appreciated that certain features of the invention which are, for clarity, described above and below in the context of separate embodiments, may also be provided in combination in a single embodiment. Conversely, various features of the invention that are, for brevity, described in the context of a single embodiment, may also be provided separately or in any sub-combination. In addition, references in the singular may also include the plural (for example, “a” and “an” may refer to one, or one or more) unless the context specifically states otherwise.

The use of numerical values in the various ranges specified in this application, unless expressly indicated otherwise, are stated as approximations as though the minimum and maximum values within the stated ranges were both proceeded by the word “about.” In this manner, slight variations above and below the stated ranges can be used to achieve substantially the same results as values within the ranges. Also, the disclosure of these ranges is intended as a continuous range including every value between the minimum and maximum values.

All patents, patent applications and publications referred to herein are incorporated by reference in their entirety.

The mono-disperse spheres, which may be used according to the invention, are able to form three-dimensional photonic crystals on the substrate surface after application on the substrate surface by arranging in a three-dimensional, tightly packed, regular and spherical (densely packed) structure on the substrate surface.

The photonic crystals produced from the mono-disperse spheres may have a mean particle size of 1 to 500 μm, preferably 100 to 5000 nm, wherein the particles may have both a platelet-like and a spherical-like structure.

Depending on the average mean particles size of the spheres, which are forming the photonic crystals according to the invention, the reflection of the wavelengths of the visible light are different from each other in a distinct manner, and the color effects are of special brilliance.

The photonic crystals, according to the invention, may be produced by providing a dispersion of mono-disperse spheres in a liquid, and then applying the dispersion onto a plain surface of an absorbent substrate so drops form on the surface, or the dispersion is deposited onto the absorbent surface as a liquid film. After removing the liquid in a short time sufficient for crystallization and solidifying, the corresponding photonic crystals are produced and may be removed from the surface by a dry or wet method.

The substrate usable in the process according to the invention is a plain absorbent material selected from the group consisting of paper, textiles and wood. Preferably, a plain paper or a plain wooden material is used, more preferably a plain paper is used, with a thickness of preferably, e.g., 10 to 500 μm.

Surprisingly, the self-assembly of a tightly packed and ordered structure of the mono-disperse spheres may be obtained by using such absorbent substrates according to the invention even if the surface of such substrates is a non-smooth surface in contrast to smooth surfaces, such as, surfaces of glass, metal or plastic substrates.

The absorbent substrate should have a liquid capacity in the range of e.g. 10 mg/m to 80 mg/mm. The liquid capacity is the ability of the substrate to absorb a liquid for a given time. Preferably the liquid capacity is in a range of e.g. 15 to 50 mg/mm. Such a substrate is able to cause a time of removing the liquid from the dispersion in a range of 30 seconds to 3 minutes, preferably less than 1 minute, at room temperature. The dispersion may be applied to the substrate surface to a thickness in a range of 500 nm to 50 μm, preferably 1 to 25 μm.

The mono-disperse spheres usable according to the invention have an average diameter of about 50 nm to about 2,000 nm. Mono-disperse spheres with a diameter of about 150 nm to about 1,500 nm are preferably used, particularly preferably with a diameter of about 200 nm to about 500 nm.

The mono-disperse spheres according to the invention may consist of almost any material if the material is able to reflect the desirable wavelengths of light.

Suitable materials for the mono-disperse spheres according to the invention include, for example, metal oxides, metal chalcogenides and silicon dioxide. The preparation of mono-disperse spheres from silicon dioxide is described in U.S. Pat. No. 4,911,903.

Mono-disperse spheres based of silicon dioxide may also be coated with non-absorbent metal oxides, such as, titanium oxide, zirconium oxide, zinc oxide, tin oxide and/or aluminium oxide, as described in U.S. Pat. No. 5,846,310 or with absorbent metal oxides such as iron oxide.

As indicated above, the mono-disperse spheres may also be based on metal oxides, such as, titanium dioxide, zirconium oxide, zinc oxide, tin oxide, aluminium oxide and mixtures thereof, as mentioned in EP-A 0644914. These may be coated with organic materials, for example silanes, as mentioned in DE-A 4316814.

Mono-disperse spheres based on polymers, for example, polystyrene, polyester, polyamides, polyurethane or poly(meth)acrylates, may also be used. Polymer spheres of this type may contain metal oxides. Mono-disperse spheres based on poly(meth)acrylates are preferably used for the process according to the invention.

Mono-disperse spheres which are produced from fluorinated (meth)acrylates, in particular, fluorinated alkyl(meth)acrylates with C2-C8 alkyl groups, are particularly suitable. Examples of such fluorinated (meth)acrylates include trifluoroethylmethacrylate, perfluoropropylmethacrylate, perfluorobutylmethacrylate, wherein the resulting polymers may also have contents of tetrafluorobutylbis(meth)acrylate or pentaerythritoltetra(meth)acrylate units in addition to fluorinated side chains. These polymers may be crosslinked, in which case they may be self-crosslinking or may be caused to crosslink by the use of crosslinking agents. Examples of crosslinking agents include cinnamoylalkyl(meth)acrylates, tetrafluorobutylbis(meth)acrylates or pentaerythritoltetra(meth)acrylates.

Mono-disperse spheres based on fluorinated, crosslinked poly(meth)acrylates are preferably used for the process according to the invention.

Polymers of this type may be produced by suspension polymerization of the monomers.

The production of a polymethacrylate from 2,2,2-trifluoroethylmethacrylate and crosslinking with pentaerythritoltetramethacrylate (PEMA) is exemplified below:

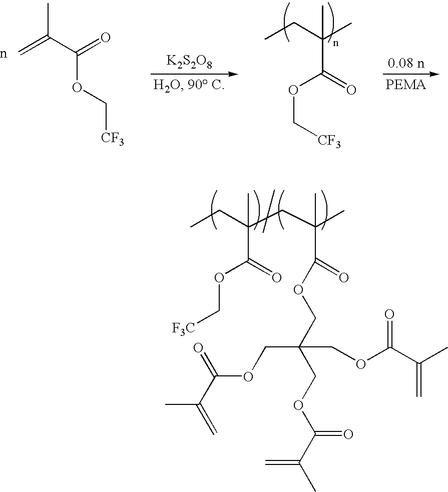

The resulted mono-disperse spheres may be purified by means of, e.g., ultrafiltration, ultracentrifugation, dialysis or ion exchange to remove unreacted monomers, small polymers, water, initiator, surfactant, agglomerated particles and the same.

The mono-disperse spheres should be substantially insoluble in the liquid usable according to the invention, and should be stably dispersed therein.

This can be accomplished, for example, by including appropriate functionality in the polymer of the mono-disperse particle. Water-dispersibility can be provided, for example, by including appropriately neutralized ionic functionality (such as, amine-neutralized carboxyl groups), and/or non-ionic soluble groups (such as, polyethylene oxide segments).

Stable dispersions can also be achieved through the use of separate dispersing agents and/or surfactants.

The liquid usable according to the invention can be an aqueous or nonaqueous vehicle, so long as the components of the liquid are compatible with the photonic crystals, i.e., do not dissolve the particles.

Aqueous vehicles may be water or a mixture of water and at least one water-soluble organic solvent (co-solvent). Representative examples of water-soluble organic solvents that may be selected are disclosed in U.S. Pat. No. 5,085,698, e.g., ethylene glycol, diethylene glycol. Preferred compositions contain 60% to 95% water, based on the total weight of the aqueous vehicle.

Nonaqueous vehicle are vehicles that are substantially comprised of a nonaqueous solvent or mixtures of such solvents, which solvents can be polar and/or nonpolar. Examples of polar solvents include alcohols, esters, ketones and ethers. Examples of nonpolar solvents include aliphatic and aromatic hydrocarbons having at least six carbon atoms and mixtures thereof including refinery distillation products and by-products.

The amount of liquid is typically in the range of preferably 30 to 99.5 wt %, more preferably from 70 to 99 wt %, based on total weight of the dispersion. Therefore the solid content of the dispersion is in the range of 1 to 90%, preferably of 20 to 60%.

An aqueous dispersion produced during production of mono-disperse polymer spheres by suspension polymerisation may be also usable as dispersion according to the invention.

The dispersion according to the invention may be applied to the substrate surface by, e.g., dipping, spraying, brushing, roll coating, flow coating or ink jet coating, to a desired thickness in a range as mentioned above.

The desired thickness may be achieved by a one-layer application or a multi-layer application of the dispersion. Preferred is a one-layer application.

The dispersion may be applied, e.g., in the form of beads by means of, e.g., spraying, such as, pneumatic or ink jet spraying, preferably, in a form of a liquid layer on the substrate surface by means of, e.g., airbrush methods.

After application of the dispersion onto the surface, a drying process follows to remove the liquid from the dispersion. This may be achieved by room temperature. The removing process may be supported by using forced air, convective and/or radiative heating, e.g., with IR radiation.

After drying the dispersion layer, the tightly packed and regularly arranged structure of the mono-disperse spheres, that means, the photonic crystals, are resulted. The crystals may be removed from the surface by dry or wet methods, such as, scraping and/or brushing, treating with ultrasound, gas or liquids, bending or folding the substrate.

The resulting photonic crystals may optionally be physically and chemically stabilized in order to obtain the structure thereof. Chemical stabilization connects the spheres of the photonic crystals by chemical modification of the surface of the spheres, for example, by the addition of soluble silicates, polymerizable aluminium compounds or curable polymer side chains, e.g., cinnamoylalkyl side chains. The surface of the spheres may also be modified in such a way that after supplying heat, thermal radiation or UV radiation, the spheres are crosslinked with one another to induce solidification of the structure.

To ensure the optical properties of the photonic crystals, the difference in the refractive indices of the mono-disperse particles and of any other components of, e.g., coating composition or ink jet ink, such as additional binder and/or additives and pigments, or of, e.g., physical or chemical stabilization, should be in a range of about 0.01 to about 2, preferably, about 0.02 to about 1.5. Optimal refractive index differences are, for example, in the range of about 0.1 to about 1.5, deviations from this are also possible.

The process according to the invention may proceed as a discontinuous process or a continuous process.

The photonic crystals are suitable for the use as pigment particles in, e.g., coating compositions. Both liquid and powder coats may be equipped with the photonic crystals according to the invention as base, intermediate or top coat. The coating compositions conventionally used in the paint industry may be used for this purpose. Water- or solvent-based coats may, for example, be used as liquid coats.

The photonic crystals may be present in a base, intermediate or top coat composition in a concentration of 0.1 to 70 weight %, preferably, in a concentration of 1 to 20 weight %, based on the coating composition.

The liquid and powder coats may be based on conventional coating binders, for example, polyester, epoxide, poly(meth)acrylate, polyamide, polycarbonate and/or polyurethane resins, aminoplastic and phenoplastic resins which may be usable together with conventional crosslinking agents. These are familiar to the person skilled in the art employed in the paint industry. The binders may also be self-crosslinking.

Water-miscible solvents or water-immiscible solvents may be used as solvents in the coating systems. Pigments, conventional paint additives, such as, plasticizers, film forming agents, fillers, thickeners, flow control agents and catalysts to accelerate crosslinking in the paint composition may also be contained.

The three-dimensional photonic crystals may also be used in a layer without coating binders and coating additives. A composition of this type may contain the photonic crystals in a concentration of 1 to 70 weight %, preferably, in a concentration of 5 to 30% weight %, based on the total composition. This composition may also contain solvents as mentioned above and/or water and additives, such as, dispersing agents and further additives, as mentioned above.

Solid preparations may also be used which contain the photonic crystals up to, for example, 95 to 99 weight %.

The coats may be applied to the substrate surface by conventional methods known in the art, with a dry coat thickness in the range of, e.g., 3 to 80 μm, then may be dried and hardened by supplying heat in an oven; by IR irradiation or electromagnetic irradiation, for example, UV radiation. Thermal curing may, for example, take place at temperatures of 20 to 140° C.

Surfaces of substrates of different types may be coated, e.g., substrates, such as, metals, plastics, wood, glass, and textiles.

The photonic crystals may be also used as pigmented particles in inks for printing purposes. The process according to the invention may be used for this purpose in such a way that the dispersion of the mono-disperse spheres may be directly used as ink composition by jetting the dispersion onto the absorbent substrate to be printed and removing the liquid by drying.

The mono-disperse spheres may be present in an ink in a concentration of from about 0.1 to about 70 wt %, preferably from about 1 to about 50 wt %, more preferably from about 1 to about 30 wt %, and particularly preferably in a concentration of from about 5 to about 20 wt %, based on the total weight of the ink jet ink composition.

Other ingredients may be formulated into the ink jet ink, to the extent that such other ingredients do not interfere with the stability and jetablity of the ink, or the mono-disperse particles, which may be readily determined by routine experimentation. Such other ingredients are in a generally well known in the art.

Inks based on aqueous vehicles can be made to be fast penetrating by including surfactants or penetrating agents, such as, glycol ethers and 1,2-alkanediols, typically in the range of from e.g. 0.01 to 15% by weight based on the total weight of the ink.

Colorants, such as, dyes or pigments usually used for ink jet ink compositions may be used in the ink, in amounts typically up to about 12%. They may be used in addition to the other components of the ink jet ink or they can be within the mono-disperse particles themselves.

Polymers may be added to the ink to improve durability (binders), such as, polystyrene, polyesters, polyamides, polyurethanes, poly(meth)acrylates and fluorinated poly(meth)acrylates, in a quantity of 0.1 to 20 wt %, based on the total weight of the ink composition. Biocides may be used to inhibit growth of microorganisms.

The inclusion of sequestering (or chelating) agents may be advantageous, for example, to eliminate deleterious effects of heavy metal impurities.

These other additives (other than vehicle, mono-disperse particles and colorants), when present, generally comprise a total of less than about 15% by weight based on the total weight of the ink.

The process according to the invention can also be used to colour the absorbent substrate as a whole.

The following examples illustrate the invention.

### EXAMPLES

### Example 1

Synthesis of Mono-Disperse Spheres

Crosslinked Polymethylmethacrylate (PMMA) was synthesized in a 2000 ml-flask with a nitrogen-inlet and a mechanical stirrer. The flask was put in a 90° C. oil bath, charged with 1200 ml deionized water and flushed with nitrogen for at least 45 min. After stopping the nitrogen-flow, 100 ml methyl methacrylate (without further purification) and 5 ml ethylene glycol dimethacrylate were added. The water-monomer mixture was stirred for 30 min to achieve temperature equilibrium. Then 40 ml of a potassium peroxodisulfate-solution (10 wt %, heated for 10 min at 90° C. under nitrogen) was added at once (<10 sec) to start the polymerization. The reaction solution was stirred vigorously for 1.5 h. After this time samples of the polymer solution showed no color change. Therefore, an almost complete conversion was assumed and the flask was opened (oxygen) to stop the polymerization. The resulted polymer particles were purified from large agglomerations by filtration through a standard paper filter. Filtration was followed by a centrifugation to remove smaller agglomerations and low molecular impurities. During centrifugation agglomeration, sediment was deposited as white solid on the bottom of the centrifugation vial. The desired polymer spheres sediment deposited as an opalescent layer on top of the first layer. Low molecular impurities like monomer and initiator salt remained in the supernatant liquid. The first sediment layer was dumped and the liquid was exchanged with water in three to four centrifugation cycles.

Average diameter of the resulting particles: 277 nm.

### Example 2

Synthesis of Dispersions of Mono-Disperse Spheres and Application

Dispersion 2.1:

2.6 vol.% of crosslinked PMMA particles, resulting from Example 1, was mixed with 10 vol. % ethylene glycol and 87.4 vol. % deionized water. The resulted dispersion was applied onto the substrates as mentioned in Table 1 by airbrush using a commercially available apparatus from the company Sil.Air to a thickness of 14 μm. The applied dispersion was dried at room temperature.

Dispersion 2.2:

10 vol. % of non-crosslinked PMMA particles resulting from Example 1 was mixed with 90 vol. % deionized water. The resulted dispersion was applied onto the substrates as mentioned in Table 1 by airbrush using a commercially available apparatus from the company Sil.Air to a thickness of 14 μm. The applied dispersion was dried at room temperature.

### Example 3

Results

TABLE 1

| | |
|---|---|
| Dispersion | Drying time [Seconds] |

| | | |
|---|---|---|
| 2.1 | paper 1 (photopaper, firm Avery): | <60 |
| | paper 2 (copy paper): | <40 |
| 2.2 | paper 1 (photopaper, firm Avery): | 130 |
| | glass: | 370 |

As it can be seen from Table 1 the drying time on the selected paper is substantially reduced in comparison to the glass substrate.

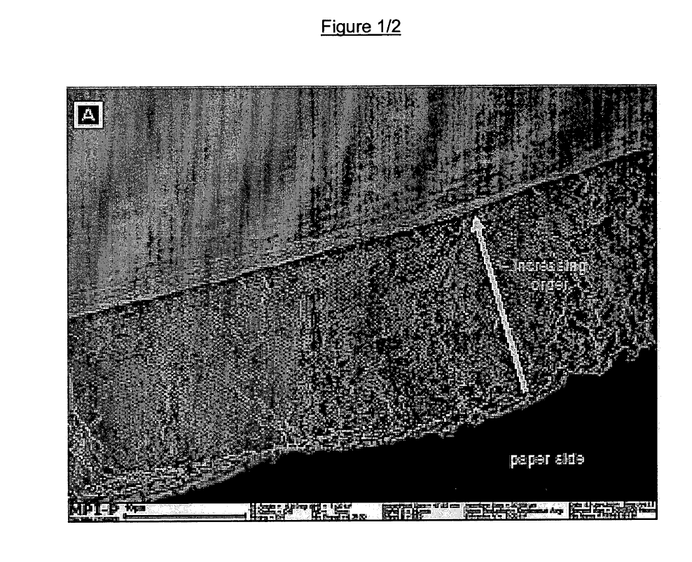

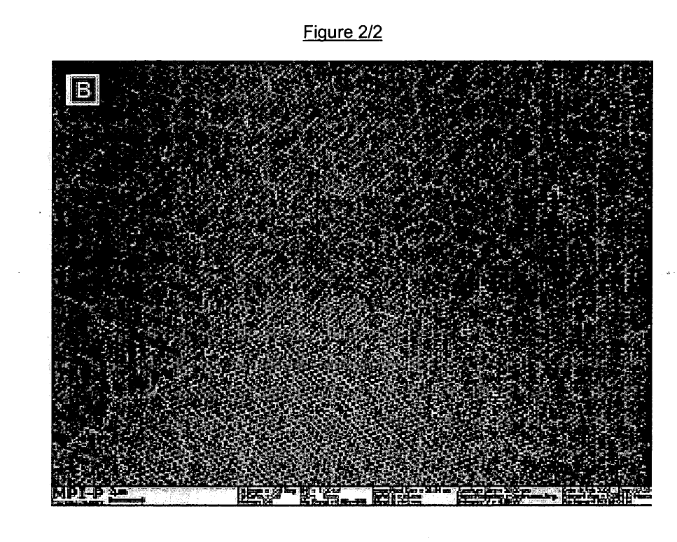

The FIGS. 1 and 2 show the resulted photonic effect using dispersion 2.1 on paper 1.

FIG. 1 shows a side view of a photonic crystal from PMMA spheres having a thickness of 14 μm prepared according to Example 2.

FIG. 2 shows a top view of photonic crystal after drying.

As it can be seen the Figures show a tightly packed and ordered structure of the mono-disperse spheres even when using a non-smooth-surface such as a paper surface.

What is claimed:

1. A process of preparation photonic crystals comprising the steps:
(a) providing a dispersion of mono-disperse spheres in a liquid,
(b) applying the dispersion onto a plain surface of an absorbent substrate selected from the group consisting of paper, textile and wood, and
(c) removing the liquid from the dispersion on the surface to produce the photonic crystals in the form of a tightly packed and regularly arranged structure of mono-disperse spheres in the resulted particles.

2. The process according to claim 1 wherein the photonic crystals have a mean particle size of 1 to 500 μm.

3. The process according to claim 1 wherein the photonic crystals have a platelet-like and/or a spherical-like structure.

4. The process according to claim 1 wherein the process is a discontinuous or a continuous process.

5. The process according to claim 1 wherein mono-disperse spheres based on polymers selected from the group consisting of polystyrene, polyester, polyamides, polyurethane and poly(meth)acrylates are used.

6. The process according to claim 5 wherein mono-disperse spheres based on fluorinated poly(meth)acrylates are used.

7. The process according to claim 1 wherein the liquid consisting of vehicles selected from the group consisting of water, alcohols, esters, ketones, ethers, hydrocarbons having at least six carbon atoms.

8. The process according to claim 1 wherein the absorbent substrate have a liquid capacity in the range of 10 mg/m to 80 mg/mm.

9. The process according to claim 1 wherein removing the liquid is done by drying.

10. The process according to claim 9 wherein the drying process is achieved by room temperature or by increased temperature.

11. The process according to claim 1 wherein the photonic crystals are removed from the surface by dry or wet methods.

12. Photonic crystals prepared according to the process of claim 1.

13. A coating composition comprising the photonic crystals according to claim 12.

14. An ink comprising the photonic crystals according to claim 12.
